(19) United States
(12) Patent Application Publication
(10) Pub. No.: US 2025/0347620 A1
(43) Pub. Date: Nov. 13, 2025

(54) PREDICTING OCTANE OF GASOLINE BLENDSTOCKS

(71) Applicant: PHILLIPS 66 COMPANY, HOUSTON, TX (US)

(72) Inventors: Ayuba Fasasi, Bartlesville, OK (US); Angel Cortes-Morales, Mesa, AZ (US); Sriram Ramaganesan, Houston, TX (US); Franklin Uba, Bartlesville, OK (US)

(73) Assignee: PHILLIPS 66 COMPANY, HOUSTON, TX (US)

(21) Appl. No.: 19/198,688

(22) Filed: May 5, 2025

Related U.S. Application Data
(60) Provisional application No. 63/643,621, filed on May 7, 2024.

(51) Int. Cl. G01N 21/359; G01N 21/3577; G01N 33/22; G01N 33/28

(52) U.S. Cl. CPC G01N 21/359; G01N 21/3577; G01N 33/22; G01N 33/2829; G01N 2201/129

(57) ABSTRACT

Blending a finished gasoline using a gasoline blending model that is derived from correlations between empirically measured octane numbers and spectral features identified in near-infrared (NIR) spectral data for a group of gasolines and gasoline subcomponents. The correlations are incorporated into generalized blend models for motor octane number and road octane number, which are incorporated into programing executed by a controller that controls the volumetric blend ratio of one or more neat gasolines and/or gasoline sub-components to produce a finished gasoline. In some embodiments, the NIR spectral data utilized for developing the model is contributed by analysis of multiple subsets of gasolines and gasoline subcomponents, where each subset is analyzed by a different NIR spectrometer.

### CROSS-REFERENCE TO RELATED APPLICATIONS

None.

### STATEMENT REGARDING FEDERALLY SPONSORED RESEARCH OR DEVELOPMENT

None.

### FIELD OF THE INVENTION

The present invention relates to a processes for blending a finished hydrocarbon fuel such as gasoline utilizing a generalized blend model that maintains product quality over time while maximizing profit and meeting all government specifications for the finished fuel.

### BACKGROUND

Product blending operations significantly impact refinery profitability and profit suffers when blended fuel products are produced that exceed government specifications for quality. Product quality giveaway refers to the lost profit opportunity that is realized by producing fuels possessing properties (e.g., octane number or volatility) that significantly surpass the government mandated specifications. Excess fuel octane represents a significant percentage of lost profit opportunity for petroleum refineries and therefore remains an opportunity to capture significant value.

Fuel blend models are used to control commercial gasoline blending and thus are integral to minimizing excess fuel octane and maximizing profit. Chemometrics has been integrated into fuel blend models by correlating one or more physical or chemical properties of a fuel blend component with its octane rating. Refineries often maintain chemometric models for different fuel grades, resulting in a collection of models for each fuel grade, each model tailored for a specific season (or ambient temperature range) that occurs throughout the year. While tailoring models to specific fuel grades can drive optimal blending and minimize fuel octane excess, maintaining the models is labor intensive, especially around transition periods between blend models when blend models often perform sub-optimally. This approach also often results in an excess of blend models at a given refinery because new models may be developed for slight changes in feed composition and/or chemistry. A large number of models makes model maintenance labor-intensive and expensive and the performance of these models often deteriorates over time. What is needed are fewer, more generalized blend models that are more broadly applicable while consistently minimizing excess octane rating of produced transportation fuel products.

### BRIEF SUMMARY OF THE DISCLOSURE

Some embodiments comprise a process for blending a finished fuel, comprising: a) analyzing a first collection of liquid hydrocarbon samples comprising multiple finished gasolines by near-infrared spectroscopy to produce a first spectral database that comprises at least one near-infrared spectrum for each finished fuel and analyzing a second collection of liquid hydrocarbon samples comprising multiple gasoline blend component streams by near-infrared spectroscopy to produce a second spectral database that comprises at least one near-infrared spectrum for each fuel blend component stream, where each near-infrared spectrum comprises spectral data comprising multiple data points; b) identifying research octane spectral features in each spectral database that comprise a subset of the spectral data that correlates with research octane number for each of the first collection and the second collection and identifying motor octane spectral features in each spectral database that comprise a subset of the spectral data that correlates with motor octane number for each of the first collection and the second collection, where the identifying results from correlating the spectral data for each member of each collection with an empirically-derived octane number for that member that is selected from road octane number and motor octane number utilizing a machine learning algorithm; c) selecting a first subset of the spectral features that best correlates with the research octane number to produce a research octane spectral features; d) selecting a second subset of the spectral features that best correlates with the motor octane number to produce a motor octane spectral features database; e) producing a first octane model that predicts research octane number for one or more gasoline blend component streams by training a first octane model algorithm on the research octane spectral features database; f) producing a second octane model that predicts motor octane number for one or more gasoline blend component streams by training a second octane model algorithm on the motor octane spectral features database; g) calculating a volumetric blend ratio comprising at least one gasoline blend component to produce a finished gasoline that meets government specifications for anti-knock index while simultaneously minimizing the difference between the anti-knock index of the finished gasoline and government specifications for minimum anti-knock index, where the calculating comprises using the first blend model and to predict the research octane number and the second blend model to predict the motor octane number for each gasoline blend component that is utilized to produce the finished gasoline.

Some embodiments additionally comprise mathematically converting the spectral data from part a) to wavelets coefficients data prior to the identifying of part b).

In some embodiments, the mathematically converting comprises decomposing the spectral data obtained from each near infrared spectrum into approximation and detail components using a mother wavelet selected from the Symlet, Haar, and Coiflets families of mother wavelets.

Some embodiments additionally comprise pre-processing the spectral data within the spectral database to produce corrected spectral data, where the pre-processing includes one or more of baseline correction, manual curation of the spectral data to remove data outliers and standardizing by removing the mean and scaling to unit variance.

In some embodiments, producing the finished gasoline is performed by a programmable logic controller, where the programmable logic controller comprises at least one processor that executes programming that incorporates the first octane model and the second octane model, where the programmable logic controller dynamically adjusts volumetric blend ratio of the multiple gasoline blend components based at least in part upon a research octane number predicted by the first octane model and a motor octane number predicted by the second octane model for each gasoline blend component utilized to produce the finished gasoline.

In some embodiments, the infrared spectroscopy comprises at least one of near infrared spectroscopy and mid-infrared spectroscopy. In some embodiments, the infrared spectrum is a near-infrared spectrum in the wavenumber range from 4000 cm⁻¹ to 4800 cm⁻¹. In some embodiments, the infrared spectrum is a near-infrared spectrum in the wavenumber range from 5500 cm⁻¹ to 6000 cm⁻¹.

In some embodiments, the selecting of the first subset of spectral features and the second subset of spectral features are each performed by a clustering data analysis algorithm. In some embodiments, the first blend model algorithm comprises a first regression algorithm and the second blend model algorithm comprises a second regression algorithm. In some embodiments, each regression algorithm is selected from Gaussian Process regression, Ridge regression and partial least squares regression.

In some embodiments, the first spectral database and the second spectral database each comprise near infrared spectrums obtained from at least two distinct near-infrared spectrometers.

In some embodiments, the identifying of research octane spectral features and motor octane spectral features comprises using a machine learning clustering algorithm to cluster the members of each of the first collection of liquid hydrocarbon samples and the second collection of liquid hydrocarbon samples into multiple pattern groups based upon spectral feature similarity, then adjusting the multiple pattern groups along the wavenumber axis to minimize differences between identified spectral features.

### BRIEF DESCRIPTION OF THE DRAWINGS

A more complete understanding of the present invention and its benefits may be acquired by referring to the description provided herein and the accompanying drawings, where:

FIG. 1 depicts a flow diagram.

FIG. 2 depicts a parity plot for a road octane number (RON) Ridge regression model.

FIG. 3 depicts a parity plot for a motor octane number (MON) Ridge regression model.

FIG. 4 depicts a parity plot for subgrade gasoline components using a generalized partial least squares (PLS) model for RON.

FIG. 5 depicts a parity plot for subgrade gasoline components using a generalized PLS model for MON.

FIG. 6 depicts a parity plot for premium gasolines using a generalized PLS model for RON.

FIG. 7 depicts a parity plot for premium gasolines using a generalized PLS model for MON.

FIG. 8 depicts a parity plot for all gasoline blend component streams using a generalized PLS model for RON.

FIG. 9 depicts a parity plot for all gasoline blend component streams using a generalized PLS model for MON.

FIG. 10 depicts combined and resampled NIR data spectrums in the wavenumber range from 5500 to 6000 cm⁻¹ and illustrates the differences in spectral data obtained from multiple NIR spectrometers.

FIG. 11 depicts in panel A two distinct spectral data clusters, and in panel B the result of horizontal shifting of the clusters on the x-axis (wave number) to minimize differences between the clusters.

FIG. 12 depicts the process of clustering and horizontal shifting for a large group of data spectrums, where the spectrums are grouped by clustering. Panel A depicts spectral data after cleaning to remove outliers (by Z-score), while panel B depicts clusters of spectral data that have been cleaned as well as horizontally-shifted to minimize the horizontal difference between clusters.

The invention is susceptible to various modifications and alternative forms, specific embodiments thereof are shown by way of example in the drawings. The drawings may not be to scale. The drawings are not intended to limit the scope of the invention to the particular embodiment illustrated.

### DETAILED DESCRIPTION

Gasoline is a complex mixture of hydrocarbons and oxygenates with variable properties, such as octane number, that form the basis of product pricing. The American Society for Testing and Materials (ASTM) standard for determining and certifying anti-knock characteristics of motor fuels utilizes standardized engines known as “knock engines” that can empirically measure both research octane number and motor octane number. However, these methods for testing octane are expensive, labor-intensive, and slow, which means they are not well-suited for directing fuel blending operations in real-time.

As an alternative, chemometric models have been developed to control fuel product octane that provide a quantitative link between chemical properties of various gasoline blend components (or subgrades) that are utilized to produce a finished gasoline and octane rating. It is common practice in commercial refineries is to maintain individual chemometric models for different gasoline “grades”, resulting in a collection of models that are tailored separately for different blending seasons. While tailoring models to specific fuel grades can drive optimal blending performance and minimize product giveaway, the task of maintaining the accuracy of such models, especially during seasonal temperature transitions, is labor intensive and sometimes requires thousands of employee hours each year. Without such maintenance, the predictive power of these multiple chemometric models deteriorates as temperatures change during the year, which increases octane giveaway.

Unfortunately, the above approach also tends to result in the existence of numerous redundant models at each refinery or blending terminal (e.g., one for each fuel product grade) given that new models may be developed for slight changes in crude oil feed compositions as well as gasoline blend component and finished product chemical composition. Utilizing a large number of models makes model maintenance tedious and more expensive, with the performance of these models prone to deteriorate over time, which increases product quality giveaway and decreases profit.

We describe herein the development of more generalized blend models that have a broader range of applicability covering many different types of gasoline blends, including multiple fuel grades. Unlike conventional schemes for producing blend models that are tailored to specific gasoline grades, the present process produces a generalized model that incorporates knowledge of both the chemical similarities in finished gasolines as well as gasoline blend components and is applicable across a wide range of octanes and blend recipes. The resulting generalized blend models simplify the task of model maintenance and provide more consistent performance over time. They also and better withstand seasonal changes to the blend requirements for finished gasolines.

In some embodiments, the process disclosed herein produces generalized blending models in part by regressing a large quantity of near infrared (NIR) spectral data obtained from analysis of both finished gasoline blends and gasoline component streams (i.e., subgrade streams) over a three-year period at a single refinery. Various chemical and mathematical linkage methods are used to generate clusters to define stream-specific models. For generalized and globalized blending models, selected finished gasoline and components deemed significant are incorporated to the clusters.

In some embodiments, the process produces globalized blending models in part by regressing NIR spectral data obtained from analysis of both finished gasoline blends and gasoline component streams at multiple refineries with the NIR spectral data obtained from each refinery by a distinct NIR spectrometers. In these embodiments, each NIR spectrometer only analyzes a portion (or subset) of the entire collection of finished gasolines and gasoline blend components obtained from all refineries. The digitized spectral data acquired for each NIR spectrum is regressed against precise knock engine measurements of the road octane number (RON) and motor octane number (MON) for each finished gasoline or gasoline component used to produce the finished gasoline. This facilitates the development of models that accurately predict either gasoline MON or gasoline RON.

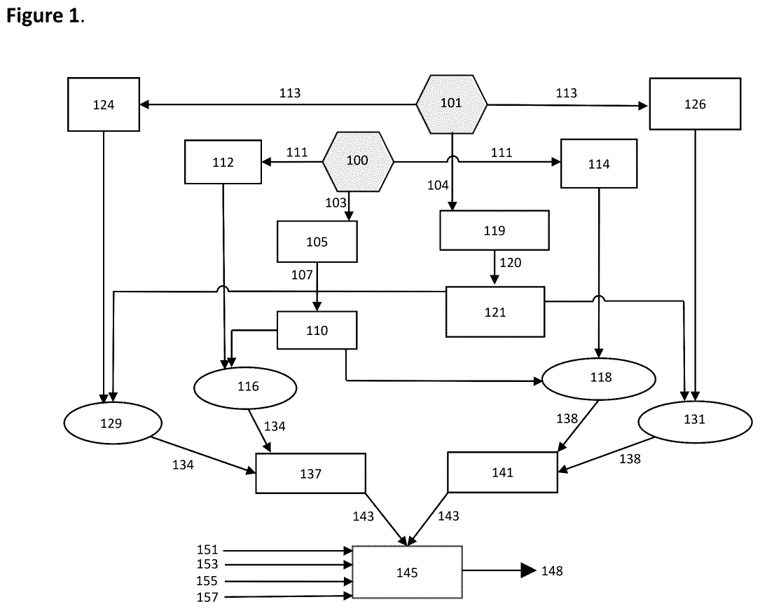

As depicted in FIG. 1, input parameters for formulating the blend model include 1) a database comprising NIR spectral data for a collection of finished gasolines and a collection of gasoline blend components and 2) empirically measured RON and MON octane values for each member of each collection. The NIR data is pre-processed using typical data standardization methods. Next, mathematical tools such as multivariate curve resolution (MCR), classical least squares (CLS), alternative least squares (ALS), multiple linear regression (MLR) and Gaussian Process Regression are used to identify and rank the most informative features in the processed gasoline spectra that best fit the model algorithm to understand concentrations and contributions from specific components. Enabled by library searching tools, interaction coefficient effects are estimated and minimized.

Each of these regressions can be described by a linear component and a residual component, where the residual estimates represent error contributed by chemical interactions within blend components as well as error contributed by the NIR instrument itself. The optical system of each IR spectrometer utilized is aligned to the He—Ne laser frequency, the error contribution from the NIR spectrometer is likely negligible for a generalized model where a single NIR spectrometer analyzes the entire collection of finished gasolines and gasoline blend components. However, globalized models incorporate spectral data obtained from multiple NIR spectrometers and the slight differences in accuracy and precision between instruments is a more significant source of error. The residual contributed by each sample varies depending on the number of components in the model. The sample residuals are a measure of the distance between each sample and the model. The size of the residuals gives an indication about the quality of fit for the model. Under ideal modeling conditions, the residual values are within (i.e., equal to or less than) the precision of the reference knock engine tests. This served as a basis for combining component and product spectra to develop a single generalized blend model for each of RON and MON within a refinery, and in some embodiments even a single global blend model for each of RON and MON that could be used across multiple refineries.

Some embodiments comprise developing a generalized blend model that utilizes as input 1) NIR spectral data obtained via a single NIR spectrometer for a collection of gasoline blend components and finished gasolines sourced from several different source refineries, and 2) reference octane values comprising motor octane number and reference octane number derived from knock engine testing of each gasoline blend component and finished gasoline in the collection.

Some embodiments comprise developing a globalized blend model that utilizes as input data 1) NIR spectral data obtained from multiple NIR spectrometers that have analyzed at least a portion of a collection comprising gasoline blend components and finished gasolines that are sourced from multiple refineries and 2) reference octane values comprising motor octane number and reference octane number derived from knock engine testing of each gasoline blend component and finished gasoline in the collection. The globalized model may be employed across multiple refineries to blend a finished gasoline due to it being NIR spectrometer agnostic and incorporating both blend components and finished gasolines from multiple refineries at distinct geographic locations.

The collection comprising finished gasolines and gasoline blend components was collected from several geographically distinct commercial refineries. The octane range for this data set spanned 69-102 research octane number (RON) and 67-93 motor octane number (MON). Near infrared spectroscopy was utilized to analyze this collection and obtain near-infrared (NIR) spectral data, typically for wavelengths in the range from 3499 to 6000 cm⁻¹, although in some embodiments, spectral data was obtained in the range from 4000 to 4800 cm⁻¹ using a flat baseline starting at 4780 cm⁻¹, while in other embodiments, spectral data was obtained an incorporated into the model in the range from 5500 to 6000 cm⁻¹. Each NIR spectrum was represented by a finite number of digital data points that typically varies from 50-5000. This data set was pre-processed in a conventional manner comprising steps such as standardization, scaling, normalization, binarization and mean removal, as needed, and converted to wavelets coefficients data. The data were then clustered into pattern groups using machine learning data clustering techniques. In some embodiments, the clustering technique utilized was selected from partitioning based (e.g., K-means), centroid-based, density-based (e.g., density-based spatial), distribution based, hierarchical (e.g., Ward), Gaussian mixtures, and any other conventional clustering technique. The clustered data were then processed to remove data outliers using conventional techniques (e.g., Z-score) and shifted horizontally (on the wavenumber axis) as needed to minimize differences between different clusters that were attributable to differences in calibration between the multiple NIR spectrometers that contributed spectral data for the model.

The resulting clustered data were utilized to train a mathematical model for the ability to accurately predict a property (e.g., RON or MON) for gasoline blend components and finished gasolines. In some embodiments, the resulting generalized (or globalized) blending model comprised a regression model selected from: Gaussian Process, Ridge Regression and Partial Least Squares (PLS) regression. Other supervised machine learning models can optionally be utilized, based upon the prediction accuracy of the model in terms of root mean squared error (RMSE) which was preferably in the range from 0.2˜0.3 and mean absolute error (MAE), which was preferably in the range from 0.1˜0.2.

The inventive process utilizes spectral data obtained from infrared spectroscopy (IR) because this technique can capture “chemical fingerprints” of hydrocarbon samples that can be correlated with a specific property of that sample, such as octane number. In some embodiments, near-infrared (NIR) spectroscopy provides excited vibrational data indicative of the overall molecular composition of each crude oil sample. Finding spectral features that correlate with properties such as octane number are impossible to identify by examination of the spectral data alone. Thus, the complexity and subtlety of these spectral signals has been an obstacle.

Certain embodiments comprise obtaining near-infrared spectral data for a sample comprising crude oil.

### PRE-PROCESSING of SPECTRAL DATA

Processing of raw spectral data was performed in a conventional manner, involving standardization, scaling, normalization, binarization, and mean removal, as needed. Mean removal is a technique that centers data by removing the average value of each characteristic, then scaling it by dividing non-constant characteristics by their standard deviation. This technique centers the data on zero and helps remove bias from features. The formula used to achieve this is: X_{scaled}=(X−mean)/std. dev. Standardization results in the rescaling of features, which in turn represents the properties of a standard normal distribution: mean=0, sd=1 In some embodiments of the globalized model, “horizontal shifting” (on the wavenumber axis) of distinct groups of clustered data was performed to minimize slight differences in the spectral data obtained from different NIR spectrometers. For these embodiments, spectral data obtained in the wavenumber range from 5500-6000 cm⁻¹ was resampled into a total of 250 datapoints for further analysis, clustered into specific groups, then horizontally shifted to minimize differences between the groups that could be attributed to slight differences in measurement accuracy and precision across different NIR spectrometers utilized to analyze the data.

### Conversion to Wavelet Coefficients Data:

Some embodiments of the inventive process in part comprise mathematical transformation of the spectral data to wavelet coefficients to enhance subtle but informative features in the data. According to wavelet theory, a discrete signal such as a spectral data point can be decomposed into “approximation” and “detail” components. Wavelet packet transform (WPT) was applied to de-noise and de-convolute digitized spectral data of hydrocarbon samples by decomposing each spectrum into coefficients (wavelet coefficients) that represent the spectrum's constituent frequencies.

Wavelet coefficients offer a different approach to removal of noise from multivariate data than other techniques such as Savitzky-Golay filtering or the fast Fourier transform. Wavelets can often enhance subtle but significant spectral features to increase the general discrimination power of the modeling approach. Using wavelets, a new set of basis vectors is developed in a new pattern space that takes advantage of the local characteristics of the data. These new basis vectors are capable of better conveying the information present in the data than axes that are defined by the original measurement variables.

In some embodiments of the present inventive process, spectral signals are “decomposed” by passing each spectrum through low-pass and high-pass scaling filters to produce a low-frequency “detail” coefficient dataset and a high-frequency “approximation” coefficient dataset. The approximation coefficients correspond to the “low-frequency signal” data in the spectra, while the detail coefficients usually correspond to the “noisy signal” portion of the data. The process of decomposition was continued with different scales of the wavelet filter pair in a step-by-step fashion to separate the noisy components from the signal until the necessary level of signal decomposition was achieved. We have found that wavelet coefficients are conducive to a variety of approaches for improving the quality of the input data for training. In some embodiments, decomposition of the data using mother wavelets from the Symlet, Haar and Coiflets wavelet families facilitated the recognition of distinct spectral features in the resulting wavelet coefficients data.

### Mathematical Data Conversion Processes

Various mathematical linkage methods are used to generate clusters to define stream specific models. Enabled by library searching tools, interaction coefficient effects are estimated and minimized. Each of these models can be described by a linear component and a constant (or residual) portion. The residual portion estimates component chemical interaction and error contribution from the NIR instrument(s). Since the optical system is aligned to the He—Ne laser frequency, the contribution from a single the NIR spectrometer is negligible for the generalized model. In some embodiments, the process produces globalized blending models in part by regressing NIR spectral data obtained from analysis of both finished gasoline blends and gasoline component streams at multiple refineries utilizing multiple NIR spectrometers. In these embodiments, each NIR spectrometer has only analyzed a portion (or subset) of the entire collection of finished gasolines and gasoline blend components.

Each sample residual varies depending on the number of components in the model. The sample residuals are a measure of the distance between each sample and the model. The size of the residuals gives an indication about the misfit of the model. Modeling conditions are preferably chosen such that the residual values are within the precision of the reference knock engine tests. This serves as a basis for combining component and product spectra to develop a single model, otherwise referred to as a generalized model.

FIG. 1 shows a flow chart outlining one embodiment of the present process. Each member of a finished gasoline collection 100 comprising multiple finished gasoline blends is analyzed by near infrared spectroscopy 103 to produce an NIR spectrum that in turn comprises spectral data, where the spectral data comprises multiple distinct digitized data points. The sum of the finished gasoline spectral data 105 obtained for all members of the finished gasoline collection 101 is then subjected to data pre-processing 107 as described herein to produced preprocessed gasoline spectral data 110.

Each member of the finished gasoline collection 100 is also subjected to knock engine analysis 111 to empirically derive its gasoline research octane number (RON) and collectively produce gasoline RON data 112 for the collection, and further, to empirically derive its gasoline motor octane number (MON) to collectively produce gasoline MON data 114 for the collection. The gasoline RON data 112 is mathematically regressed against the pre-processed gasoline spectral data 110 to produce gasoline RON regression data 116. The gasoline MON data 114 is mathematically regressed against the pre-processed gasoline spectral data 110 to produce gasoline MON regression data 118.

Each member of a gasoline blend component collection 101 comprising multiple subgrade gasoline streams (or multiple subgrade streams) is analyzed by near infrared spectroscopy 104 to produce an NIR spectrum that in turn comprises spectral data, where the spectral data comprises multiple distinct digitized data points. The sum of the gasoline blend component spectral data 119 obtained for all members of the gasoline blend component collection 101 is then subjected to data pre-processing 120 as described herein to produced preprocessed blend component spectral data 121.

Each member of the gasoline blend component collection 101 is also subjected to knock engine analysis 113 to empirically derive its blend component research octane number (RON) to collectively produce blend component RON data 124 and a blend component MON data (MON) 126. The blend component RON Data 124 is regressed against preprocessed component spectral data 121 to produce components RON regression data 129. The blend component MON data 126 is regressed against preprocessed component spectral data 121 to produce components MON regression data 131.

The gasoline RON regression data 116 and the components RON regression data 129 are each curated 134 to select informative features within the data (i.e., regions for which the correlation with octane number is particularly strong) that are incorporated into a trained RON blend model 137. The gasoline MON regression data 118 and the components MON regression data 131 are each curated 136 to select informative features within the data (i.e., regions for which the correlation with octane number is particularly strong) that are incorporated into a trained MON blend model 141. The algorithms comprising the trained RON blend model 137 and the trained MON blend model 141 are each incorporated as programming 143 that when executed, operates a programmable linear controller 145 that is capable of implementing each blend model to produce a finished gasoline 148 that meets all government specifications for Anti-Knock Index (AKI) by controlling the operation of one or more adjustable valves in one or more pipes (not depicted), each pipe containing a blend component of a finished gasoline (e.g., a gasoline subgrade or subcomponent 151, a neat gasoline 153, ethanol 155, butane 157, etc). The term AKI as used herein is given its standard definition, which is the sum of (RON+MON) divided by 2.

FIG. 1 shows a flow chart outlining one embodiment of the present process. Each member of a finished gasoline collection 100 comprising multiple finished gasoline blends is analyzed by near infrared spectroscopy 103 to produce an NIR spectrum that in turn comprises spectral data, where the spectral data comprises multiple distinct digitized data points. The sum of the finished gasoline spectral data 105 obtained for all members of the finished gasoline collection 101 is then subjected to data pre-processing 107 as described herein to produced preprocessed gasoline spectral data 110.

Each member of the finished gasoline collection 100 is also subjected to knock engine analysis 111 to empirically derive its gasoline research octane number (RON) and collectively produce gasoline RON data 112 for the collection, and further, to empirically derive its gasoline motor octane number (MON) to collectively produce gasoline MON data 114 for the collection. The gasoline RON data 112 is mathematically regressed against the pre-processed gasoline spectral data 110 to produce gasoline RON regression data 116. The gasoline MON data 114 is mathematically regressed against the pre-processed gasoline spectral data 110 to produce gasoline MON regression data 118.

Each member of a gasoline blend component collection 101 comprising multiple subgrade gasoline streams (or multiple subgrade streams) is analyzed by near infrared spectroscopy 104 to produce an NIR spectrum that in turn comprises spectral data, where the spectral data comprises multiple distinct digitized data points. The sum of the gasoline blend component spectral data 119 obtained for all members of the gasoline blend component collection 101 is then subjected to data pre-processing 120 as described herein to produced preprocessed blend component spectral data 121.

Each member of the gasoline blend component collection 101 is also subjected to knock engine analysis 113 to empirically derive its blend component research octane number (RON) to collectively produce blend component RON data 124 and a blend component MON data (MON) 126. The blend component RON Data 124 is regressed against preprocessed component spectral data 121 to produce components RON regression data 129. The blend component MON data 126 is regressed against preprocessed component spectral data 121 to produce components MON regression data 131.

The gasoline RON regression data 116 and the components RON regression data 129 are each curated 134 to select informative features within the data (i.e., regions for which the correlation with octane number is particularly strong) that are incorporated into a trained RON blend model 137. The gasoline MON regression data 118 and the components MON regression data 131 are each curated 136 to select informative features within the data (i.e., regions for which the correlation with octane number is particularly strong) that are incorporated into a trained MON blend model 141. The algorithms comprising the trained RON blend model 137 and the trained MON blend model 141 are each incorporated as programming 143 that when executed, operates a programmable linear controller 145 that is capable of implementing each blend model to produce a finished gasoline 148 that meets all government specifications for Anti-Knock Index (AKI) by controlling the operation of one or more adjustable valves in one or more pipes (not depicted), each pipe containing a blend component of a finished gasoline (e.g., a gasoline subgrade or subcomponent 151, a neat gasoline 153, ethanol 155, butane 157, etc). The term AKI as used herein is given its standard definition, which is the sum of (RON+MON) divided by 2.

FIG. 1 shows a flow chart outlining one embodiment of the present process. Each member of a finished gasoline collection 100 comprising multiple finished gasoline blends is analyzed by near infrared spectroscopy 103 to produce an NIR spectrum that in turn comprises spectral data, where the spectral data comprises multiple distinct digitized data points. The sum of the finished gasoline spectral data 105 obtained for all members of the finished gasoline collection 101 is then subjected to data pre-processing 107 as described herein to produced preprocessed gasoline spectral data 110.

Each member of the finished gasoline collection 100 is also subjected to knock engine analysis 111 to empirically derive its gasoline research octane number (RON) and collectively produce gasoline RON data 112 for the collection, and further, to empirically derive its gasoline motor octane number (MON) to collectively produce gasoline MON data 114 for the collection. The gasoline RON data 112 is mathematically regressed against the pre-processed gasoline spectral data 110 to produce gasoline RON regression data 116. The gasoline MON data 114 is mathematically regressed against the pre-processed gasoline spectral data 110 to produce gasoline MON regression data 118.

Each member of a gasoline blend component collection 101 comprising multiple subgrade gasoline streams (or multiple subgrade streams) is analyzed by near infrared spectroscopy 104 to produce an NIR spectrum that in turn comprises spectral data, where the spectral data comprises multiple distinct digitized data points. The sum of the gasoline blend component spectral data 119 obtained for all members of the gasoline blend component collection 101 is then subjected to data pre-processing 120 as described herein to produced preprocessed blend component spectral data 121.

Each member of the gasoline blend component collection 101 is also subjected to knock engine analysis 113 to empirically derive its blend component research octane number (RON) to collectively produce blend component RON data 124 and a blend component MON data (MON) 126. The blend component RON Data 124 is regressed against preprocessed component spectral data 121 to produce components RON regression data 129. The blend component MON data 126 is regressed against preprocessed component spectral data 121 to produce components MON regression data 131.

The gasoline RON regression data 116 and the components RON regression data 129 are each curated 134 to select informative features within the data (i.e., regions for which the correlation with octane number is particularly strong) that are incorporated into a trained RON blend model 137. The gasoline MON regression data 118 and the components MON regression data 131 are each curated 136 to select informative features within the data (i.e., regions for which the correlation with octane number is particularly strong) that are incorporated into a trained MON blend model 141. The algorithms comprising the trained RON blend model 137 and the trained MON blend model 141 are each incorporated as programming 143 that when executed, operates a programmable linear controller 145 that is capable of implementing each blend model to produce a finished gasoline 148 that meets all government specifications for Anti-Knock Index (AKI) by controlling the operation of one or more adjustable valves in one or more pipes (not depicted), each pipe containing a blend component of a finished gasoline (e.g., a gasoline subgrade or subcomponent 151, a neat gasoline 153, ethanol 155, butane 157, etc). The term AKI as used herein is given its standard definition, which is the sum of (RON+MON) divided by 2.

The following examples of certain embodiments of the invention are given. Each example is intended to illustrate a specific embodiment, but the scope of the invention is not intended to be limited to the embodiments specifically disclosed.

### Example

A first collection was obtained comprising samples of finished gasolines obtained from a single refinery over a three-year period. A second collection comprising various gasoline blends components (i.e., subgrade blend components) was obtained at various locations within a refinery over the same time period. The octane range for this data set spanned 69-102 research octane number (RON) and 67-93 motor octane number (MON). Knock engine testing was performed using a cooperative fuel research engine according to ASTM Procedures ASTM D2699 (for RON) and D2700 (for MON) using a statistical quality control chart.

Near infrared (NIR) spectroscopy was performed in a conventional manner using an ABB Bomen FT-NIR spectrometer equipped with indium arsenide detector. Samples were scanned in a fixed 0.5 mm flow-through cell with sapphire windows. The spectral resolution of the instrument is 4 cm−1, and the number of sample and background scans was 32. This analyzer was utilized to analyze each member of each collection to obtain NIR spectral data in the wavelength range from 4000 cm⁻¹ to 4800 cm⁻¹.

Data preprocessing is crucial in developing robust and reliable models from optical spectroscopic data by avoiding “masking” of bad data points and “swamping” of good data points by bad data points. Each NIR spectrum was pre-processed by vector normalization to unit length to addresses minor differences in optical pathlength that may exist between laboratory and online spectrometers. All NIR spectra were then auto scaled to remove inadvertent weighting of the data that would otherwise occur due to differences in magnitude across measurement variables.

### Modeling Methodology

Instead of classifying the data by streams for modeling purposes, a hierarchical classification (HCA) scheme based on Ward's method using square Euclidian distances was used to identify important classes (or clusters) within the spectral data. Such techniques are conventional in nature but in this instance identified that five classes could account for more than 90% of the variance in the data and seven classes could account for 97% of the variance in the data.

This data set comprising selected sub-regions of the pre-processed spectral data regressed against measured octane values for each gasoline stream (or subgrade stream) was used to train, test, and validate models that could predict the MON or RON of a gasoline stream (or subgrade stream) based upon its NIR spectrum. The octane range for this data set spanned 69-102 RON and 67-93 MON. This data set was used to train, test, and validate two types of regression models: Ridge Regression and Partial Least Squares (PLS) regression models. The Ridge regression model was built using scikit-learn, an open source Python package for developing machine learning models. The PLS model was built by first using MATLAB® and UNSCRAMBLER® to determine optimal model parameters and subsequently using AIRS® and GRAMS® to develop a deployable version of the model compatible with a typical refinery blend control system. The end result was two generalized octane prediction models-one for neat gasoline RON and another for neat gasoline MON.

Once trained, an influence plot (i.e., a residual plot as a function of multivariate distance of each observation from the center of the data set) was used to identify datapoints that were likely to cause swamping or masking and were not identified during the exploratory data analysis. Such datapoints would fit perfectly into the model but have little predictive power. The results of this influence plot showed a tight clustering of all data points in the lower left quadrant, indicating there were likely no outliers present to cause inaccuracy.

Certain embodiments comprise manually curating potential differentiating features in subsets of the data that are identified by the regression. This approach focuses on potential differentiating features with the highest probability of informing the correlation between the recognized features and octane number.

Potential differentiating data features that are recognized by regression of subsets of the spectral data with known octane number for each sample are then subjected to manual curation Manual curation of potential differentiating data features comprises eliminating from consideration any potential differentiating data features that are deemed by either a process operator or an automated curation process to have a high probability of not being highly correlated with the octane number of finished gasolines or sub-components thereof. Potential differentiating data features most likely to be subject to manual curation typically are located in a region of the spectral data where the data is typically characterized by a low signal to noise ratio.

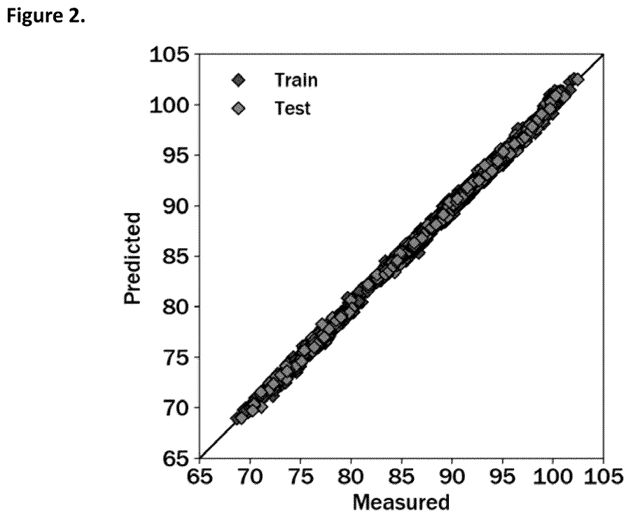

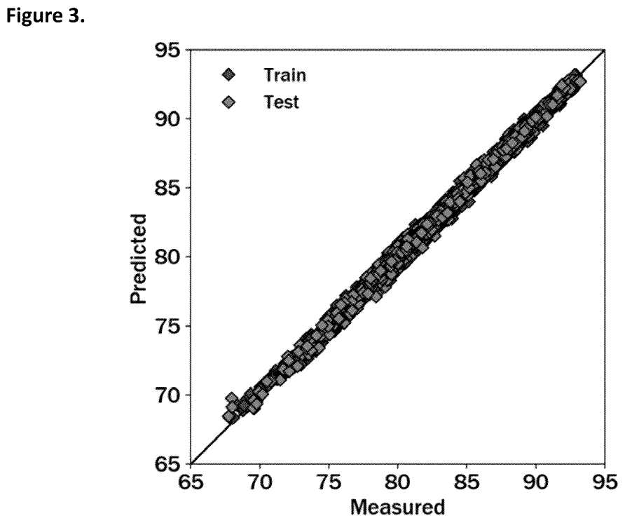

Once developed, the MON and RON neat gasoline models were validated utilizing additional unseen spectral data and empirically derived octane number data that spanned two seasonal transitions from spring to summer and summer to fall at a commercial refinery. FIG. 2 and FIG. 3 show parity plots for training and testing of the Ridge regression RON and MON models, respectively. These generalized models had root mean square error (RSME) of approximately one-third of an octane number or less. When validated against conventional “stream-specific” blend models the generalized Ridge blend models performed as good or better than the stream-specific blend models, even after segregating the data by streams. Table 1 shows the performance of the Ridge Model for all gasoline grades and subgrades tested. Each table value represents the calculated RSME (units are octane number).

TABLE 1
Validation RSME for Generalized Blend
Models developed using Ridge Regression

| | | | Ridge | | Ridge |
|---|---|---|---|---|---|
| | Sample | Conventional | RON | Conventional | MON |
| Stream | Number | RON Model | Model | MON Model | Model |
| Premium | 15 | 0.28 | 0.28 | 0.64 | 0.29 |
| Subgrades | 120 | 0.85 | 0.32 | 0.92 | 0.60 |
| Reformate | 163 | 0.78 | 0.30 | 0.67 | 0.42 |
| Raw | 46 | 0.34 | 0.31 | 0.41 | 0.31 |
| gasoline | | | | | |
| Alkylate | 20 | 0.63 | 0.24 | 0.64 | 0.31 |
| C5 KP | 43 | 1.58 | 0.68 | 2.57 | 1.13 |

Using similar methodology, the data were again regressed using partial least squares (PLS) regression instead of Ridge regression. The results for PLS models were found to be nearly identical to those for Ridge models, yielding training and testing RMSE of 0.30 octane number for RON and 0.35 octane number for MON, respectively.

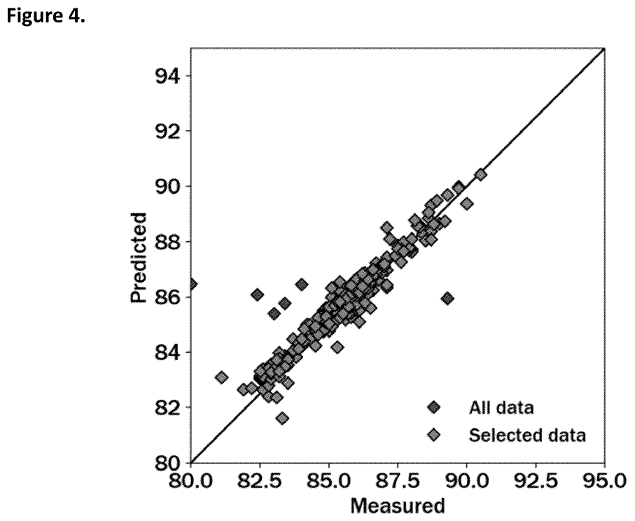

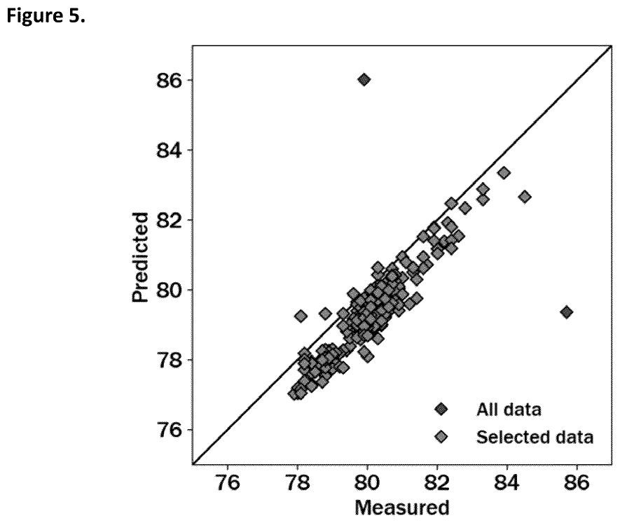

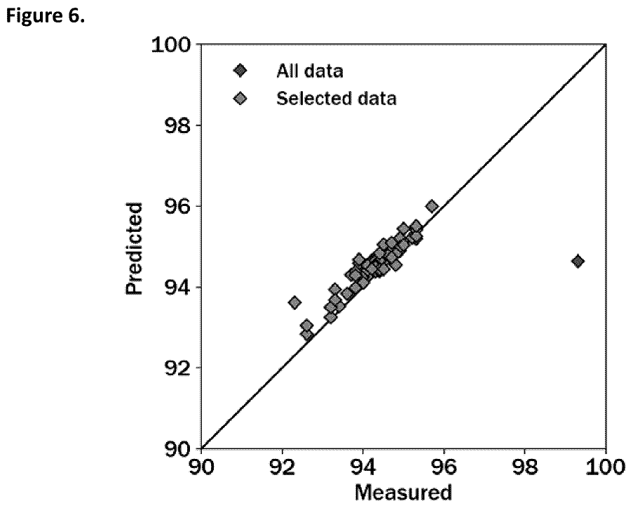

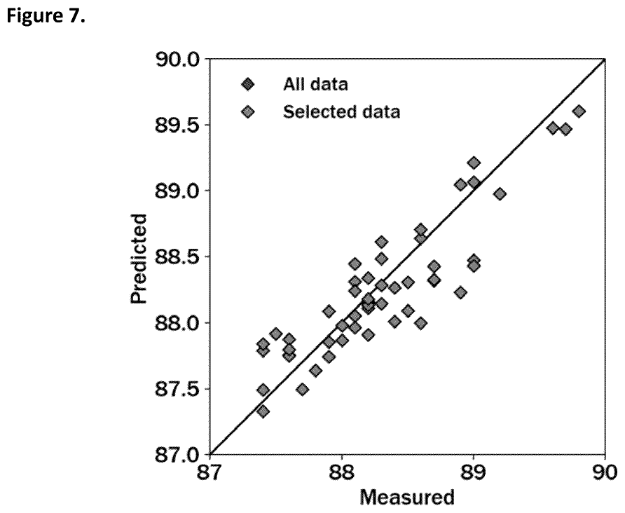

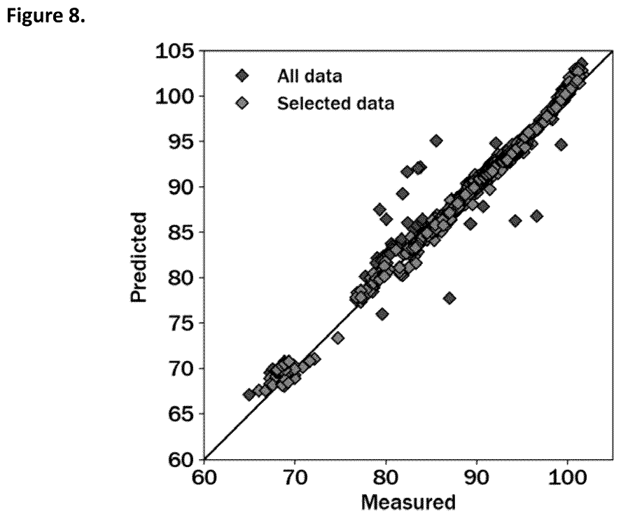

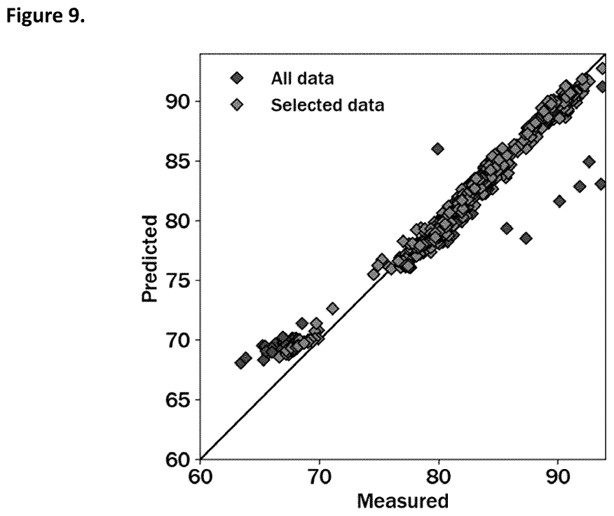

Further demonstrating the process, a parity plot predicting RON for subgrade gasoline is shown in FIG. 4. Diamonds denote samples where the predicted RSME in the octane number estimate was less than 2 and circles denote samples for which the predicted RSME was greater than 2. The parity plot predicting MON for subgrade gasoline is shown in FIG. 5 with similar markings for data where the RSME for the predicted octane value was less than 2. The parity plot predicting RON for premium grade gasoline is shown in FIG. 6. The resulting parity plot predicting MON for premium grade gasoline is shown in FIG. 7. The resulting parity plot predicting RON for all grades of gasoline is shown in FIG. 8. The resulting parity plot predicting RON for all grades of gasoline is shown in FIG. 9.

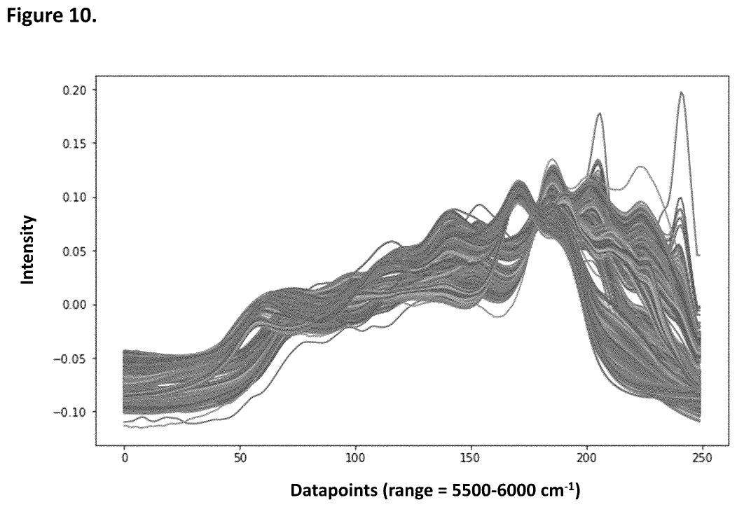

FIG. 10 depicts combined and resampled NIR data spectrums in the wavenumber range from 5500 to 6000 cm⁻¹ and illustrates typical differences in the spectral data obtained from multiple NIR spectrometers. These differences may be due to small differences in the photo sensitivity between multiple spectrometers or small differences in calibration.

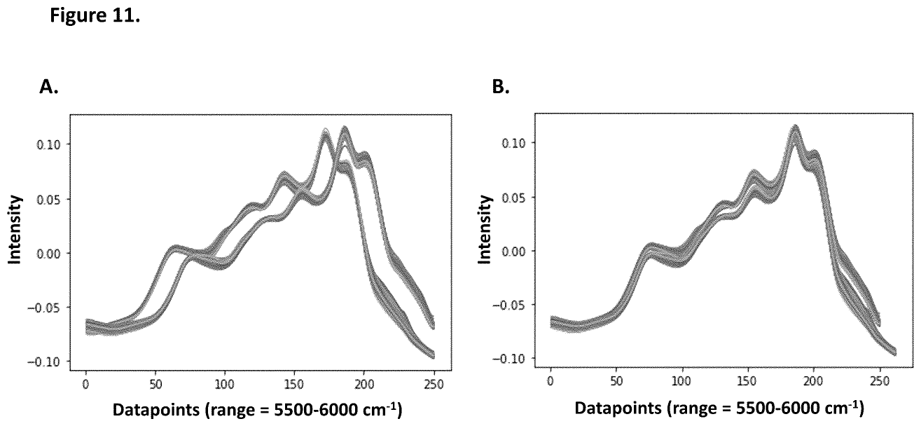

FIG. 11 depicts in panel A two distinct spectral data clusters identified via clustering algorithm. Panel B shows the result of horizontal shifting two identified clusters on the x-axis (corresponding to wave number) to minimize differences in intensity (Y-axis) between the clusters.

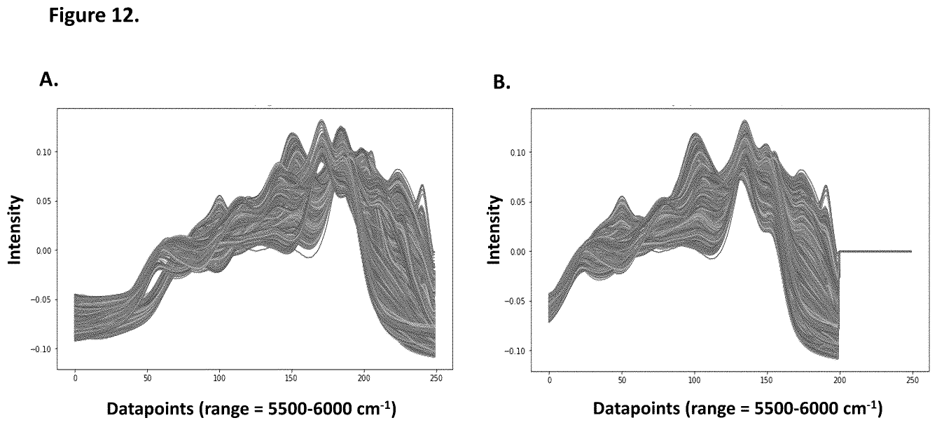

FIG. 12 depicts the process of clustering and horizontal shifting for a large group of data spectrums, where the spectrums are grouped by clustering. Panel A depicts spectral data after cleaning to remove outliers (by Z-score), while panel B depicts clusters of spectral data that have been cleaned as well as horizontally-shifted to minimize the horizontal difference between clusters. These steps allow the spectral data obtained from multiple NIR spectrometers to be combined and utilized to produce a globalized model that can be utilized across multiple gasoline blending sites, as long as each gasoline blending site contributed spectral data that contributed to the models (e.g., models for RON. MON or other useful properties of a neat gasoline or gasoline blend component).

The invention is susceptible to various modifications and alternative forms, specific embodiments thereof are shown by way of example in the drawings. The drawings may not be to scale. The drawings are not intended to limit the scope of the invention to the particular embodiment illustrated.

Although the systems and processes described herein have been described in detail, various changes, substitutions, and alterations can be made without departing from the spirit and scope of the invention as delineated by the following claims. Further, the description, abstract and drawings are not intended to limit the scope of the claims only to the embodiments disclosed.

We claim:

1. A process for blending a finished fuel, comprising:
a) analyzing a first collection of liquid hydrocarbon samples comprising multiple finished gasolines by near infrared spectroscopy to produce a first spectral database that comprises at least one near-infrared spectrum for each finished fuel and analyzing a second collection of liquid hydrocarbon samples comprising multiple gasoline blend component streams by near-infrared spectroscopy to produce a second spectral database that comprises at least one near-infrared spectrum for each fuel blend component stream, wherein each near-infrared spectrum comprises spectral data comprising multiple data points;
b) identifying research octane spectral features in each spectral database that comprise a subset of the spectral data that correlates with research octane number for each of the first collection and the second collection and identifying motor octane spectral features in each spectral database that comprise a subset of the spectral data that correlates with motor octane number for each of the first collection and the second collection,
wherein the identifying results from correlating the spectral data for each member of each collection with an empirically-derived octane number for that member that is selected from road octane number and motor octane number utilizing a machine learning algorithm;
c) selecting a first subset of the spectral features that best correlates with the research octane number to produce a research octane spectral features database;
d) selecting a second subset of the spectral features that best correlates with the motor octane number to produce a motor octane spectral features database;
e) producing a first blend model algorithm that predicts research octane number for gasoline blend component streams by training the first blend model algorithm on the research octane spectral features database;
f) producing a second blend model algorithm that predicts motor octane number for one or more gasoline blend component streams by training the second blend model algorithm on the motor octane spectral features database;
g) calculating a volumetric blend ratio comprising at least one gasoline blend component to produce a finished gasoline that meets government specifications for anti-knock index while simultaneously minimizing the difference between the anti-knock index of the finished gasoline and government specifications for minimum anti-knock index, wherein the calculating comprises using the first blend model and the second blend model to predict the research octane number and the motor octane number for each gasoline blend component that is utilized to produce the finished gasoline.

2. The process of claim 1, additionally comprising mathematically converting the spectral data from part a) to wavelets coefficients data prior to the identifying of part b).

3. The process of claim 2, wherein the mathematically converting comprises decomposing the spectral data obtained from each near infrared spectrum into approximation and detail components using a mother wavelet selected from the Symlet, Haar, and Coiflets families of mother wavelets.

4. The process of claim 1, additionally comprising pre-processing the spectral data within the spectral database to produce corrected spectral data, wherein the pre-processing includes one or more of baseline correction, manual curation of the spectral data to remove data outliers and standardizing by removing the mean and scaling to unit variance.

5. The process of claim 1, wherein producing the finished gasoline is performed by a programmable logic controller, wherein the programmable logic controller comprises at least one processor that executes programming that incorporates the first blend model algorithm and the second blend model algorithm, wherein the programmable logic controller dynamically adjusts volumetric blend ratio of the multiple gasoline blend components based at least in part upon the predicted research octane number and the predicted motor octane number of each gasoline blend component to produce the finished gasoline.

6. The process of claim 1, wherein the infrared spectroscopy comprises at least one of near infrared spectroscopy and mid-infrared spectroscopy.

7. The process of claim 6, wherein the infrared spectrum is a near-infrared spectrum in the wavenumber range from 4000 cm⁻¹ to 4800 cm⁻¹.

8. The process of claim 6, wherein the infrared spectrum is a near-infrared spectrum in the wavenumber range from 5500 cm⁻¹ to 6000 cm⁻¹.

9. The process of claim 1, wherein the selecting of the first subset of spectral features and the second subset of spectral features are each performed by a clustering data analysis algorithm.

10. The process of claim 1, wherein the first blend model algorithm comprises a first regression algorithm and the second blend model algorithm comprises a second regression algorithm.

11. The process of claim 10, wherein each regression algorithm is selected from Gaussian Process regression, Ridge regression and partial least squares regression.

12. The process of claim 1, wherein the first spectral database and the second spectral database each comprise near infrared spectrums obtained from at least two distinct near-infrared spectrometers.

13. The process of claim 1, wherein the identifying of research octane spectral features and motor octane spectral features comprises using a machine learning clustering algorithm to cluster the members of each of the first collection of liquid hydrocarbon samples and the second collection of liquid hydrocarbon samples into multiple pattern groups based upon spectral feature similarity, then adjusting the multiple pattern groups along the wavenumber axis to minimize differences between identified spectral features.
